(19) United States
(12) Patent Application Publication
(10) Pub. No.: US 2009/0112021 A1
(43) Pub. Date: Apr. 30, 2009

(54) Preparation of cyclopropyl sulfonylamides

(75) Inventor: Stefan Hildbrand, Moehlin (SZ)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/288,897

(22) Filed: Oct. 24, 2008

(30) Foreign Application Priority Data
Oct. 25, 2007 (EP) 07119267.8

(51) Int. Cl. C07C 311/00

(52) U.S. Cl. 564/80

(57) ABSTRACT

A novel process for the preparation of cyclopropyl sulfonamide of the formula I is described. Cyclopropyl sulfonamide is a versatile building block for many biologically active compounds.

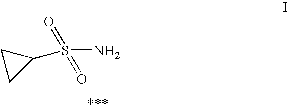

### CROSS REFERENCE TO PRIOR APPLICATIONS

This application claims the benefit of priority to EP 07119267.8 filed Oct. 25, 2007 the contents of which are hereby incorporated in their entirety by reference.

### FIELD OF THE INVENTION

The invention comprises a novel process suitable for large scale manufacture of cyclopropyl sulfonamide (I). Cyclopropyl sulfonamide is a versatile building block for many biologically active compounds (J. Li et al., Synlett 2006, 725-728).

### SUMMARY OF THE INVENTION

The invention provides a process which comprises the steps of:

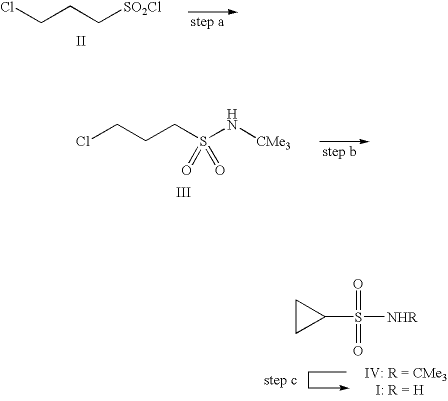

(i) contacting chloropropane sulfonyl chloride (II) with N-tert-butyl amine and a base in a first non-polar solvent to afford N-tert-butyl-(3-chloro) propyl sulfonamide (III);

(ii) contacting III with a n-alkyl lithium in a second no-polar solvent to cyclize the 3-chloro-group and afford IV;

(iii) contacting IV with acid, while passing inert gas through the reaction, to cleave the tert-butyl group and afford I;

wherein the process is performed without the isolation of III and IV.

### DETAILED DESCRIPTION OF THE INVENTION

The invention provides a process which does not requires isolation or purification of intermediates and therefore is more cost efficient and which further avoids the use of environmentally undesirable trifluoroacetic acid in the cleavage of the tert-butyl moiety.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

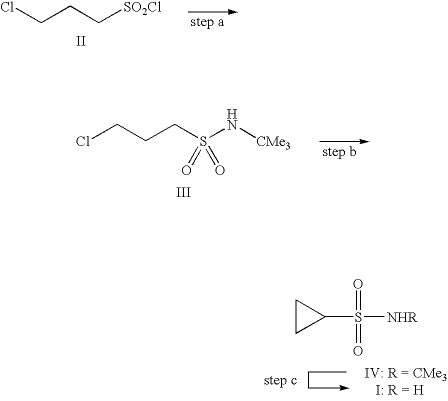

The compound can be prepared by a three step synthesis comprising a) the conversion of chloropropane sulfonyl chloride of the formula with tert-butyl amine to form the N-tert-butyl-(3-chloro) propyl sulfonamide of the formula; (b) the subsequent ring closing of the N-tert-butyl-(3-chloro) propyl sulfonamide of the formula III with n-butyl lithium to form the cyclopropane sulfonic acid tert-butylamide (IV) and finally (c) cleaving the tert-butyl group with trifluoroacetic acid (J. Li et al., Synlett 2006, No. 5, 725-728; PCT Publ. WO 2006/086381).

The current synthesis requires the isolation of the intermediates of step a) and b) the N-tert-butyl-(3-chloro) propyl sulfonamide (III) and the cyclopropane sulfonic acid tert-butylamide (IV). Furthermore the cleavage of the tert-butyl group with a large excess trifluoroacetic acid is under an environmentally undesirable and an efficient manufacturing process will minimize the number of unit operations. Thus the currently disclosed synthesis is not optimized for large scale manufacture.

The object of the present invention was therefore to find a process alternative which does not require the isolation of the intermediate products and which is able to replace the trifluoroacetic acid by a more environment friendly alternative.

The process for the preparation of cyclopropyl sulfonamide of the formula comprises (a) contacting chloropropane sulfonyl chloride (I) with tert-butyl amine to form the N-tert-butyl-(3-chloro) propyl sulfonamide (II);

(b) ring closure of III with a n-alkyl lithium to afford IV; and finally,

(c) cleaving the tert-butyl group in the presence of an acid to afford (I) whereby the process is characterized in that it is performed without the isolation of the intermediate N-tert-butyl-(3-chloro) propyl sulfonamide (III) and cyclopropane sulfonic acid tert-butylamide (IV).

Step (a) comprises the conversion of chloropropane sulfonyl chloride (II) with tert-butyl amine to form the N-tert-butyl-(3-chloro) propyl sulfonamide (III). The reaction is usually performed in the presence of an non-polar organic solvent which is imiscible or only poorly miscible with water, such as in methyltetrahydrofuran, tert-butyl methyl ether or toluene, preferably in toluene.

The formation of III could be achieved with a substantial excess of tert-butyl amine. However, it was found preferable to use smaller quantities of tert-butyl amine and add triethylamine as the base to sequester the HCl liberated during the reaction. The conversion in step (a) is commonly carried out at a reaction temperature of −50° C. to 40° C., preferably of −10° C. to 20° C.

Upon completion of the amide formation the reaction mixture can then be treated with aqueous hydrochloric acid. The organic layer can be separated from the aqueous layer and after washing the organic layer with water and subsequent azeotropic removal of water, the sulfonamide III can be made available for step (b) as a solution in the respective organic solvent, preferably in toluene. The solution can be concentrated prior to step (b) if desired

Step (b) comprises the ring closure of the N-tert-butyl-(3-chloro) propyl sulfonamide (III) with a n-alkyl lithium to form the cyclopropane sulfonic acid tert.-butylamide (IV). Tetrahydrofuran is added followed by at least two equivalents of n-alkyl lithium. The n-alkyl lithium base is selected from n-butyl lithium or n-hexyl lithium, preferably n-butyl lithium. The conversion in step (b) is thus performed in a solvent mixture of tetrahydrofuran and toluene in a ratio of approx. 3:1 at a reaction temperature of −70° C. to 0° C., preferably to −50° C. to −20° C.

Upon completion of the reaction the reaction mixture is usually warmed to ambient temperature and quenched with water. The cyclopropane sulfonic acid tert.-butylamide (IV) can be made available for step (c) in the form of a toluene/tetrahydrofuran solution after separation of the organic layer from the aqueous layer and after washing the organic layer with water.

Step (c) comprises the cleaving the tert-butyl group in the presence of an acid to form the cyclopropyl sulfonamide (I). The solution of cyclopropane sulfonic acid tert.-butylamide obtained from step (b) is concentrated by distillation then expediently treated with formic acid, or with aqueous formic acid, at a reaction temperature of 60° C. to 100° C., preferably 70° C. to 90° C. until the conversion is completed. In order to achieve a complete conversion it is mandatory to steadily bubble an inert gas through the mixture during the entire reaction.

Isolation and purification of the cyclopropyl sulfonamide (I) can carried out by removal of residual amounts of formic acid by co-evaporation with toluene and subsequent crystallization of the residue with a mixture of toluene and ethanol. A ratio toluene to ethanol of >3:1 was found to be preferable.

The processes of the present invention affords cyclopropyl sulfonamide (I) in an overall yield of 70-75% and with an assay of min. 99% (area).

The term “aprotic” or “nonpolar” solvent means organic solvents such as diethyl ether, ligroin, pentane, hexane, cyclohexane, heptane, chloroform, benzene, toluene, dioxane, tetrahydrofuran, dichloromethane or ethyl acetate.

As used herein, the term “treating”, “contacting” or “reacting” when referring to a chemical reaction means to add or mix two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). The starting materials and reagents used in preparing these compounds generally are either available from commercial suppliers, such as Aldrich Chemical Co., or are prepared by methods known to those skilled in the art following procedures set forth in references. Materials, reagents and the like to which reference are made in the following description and examples are obtainable from commercial sources, unless otherwise noted. General synthetic procedures have been described in treatise such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40 and will be familiar to those skilled in the art.

### EXAMPLE 1

To a solution of 36.6 g (0.50 mol) tert-butylamine and 50.4 g (0.50 mol) triethylamine in 400 mL of toluene cooled to 0° C. to 5° C. was added 3-chloropropane sulfonyl chloride (73.0 g, 0.41 mol) within 30 to 60 minutes and the resulting mixture was stirred at 5° C. for 10 min. The mixture was warmed to room temperature and treated with 200 mL of 1 M hydrochloric acid. The layers were allowed to separate and the aqueous layer was removed. The organic layer was washed with water (1×100 mL). From the organic layer approx. 250 mL of toluene were distilled off. THF (700 mL) was added and distillation was continued until the residual volume in the reaction vessel was approx. 600 mL. The mixture was cooled to −30° C. and 385 g of BuLi (15% in hexane, 2.2 eq) was added. After 30 min at −30° C. the mixture was warmed to 0° C. treated with water (200 mL). The layers were allowed to separate. The aqueous layer was removed and the organic layer was washed with water (1×100 mL) and concentrated. The residue was treated with formic acid (300 mL) at 80° C. for 20 h. During the entire reaction a slight stream of nitrogen was bubbled through the solution. After complete conversion the mixture was concentrated to dryness. Residual amounts of formic acid were removed by co-evaporation with toluene (2×250 mL). The remaining residue was treated with toluene (100 mL) and ethanol (35 mL), heated to 70 to 75° C. and stirred at this temperature until a clear solution was obtained. The solution was cooled to 50° C. and treated at this temperature with 300 mL of toluene. The mixture was then cooled to −10 to −15° C. and stirred at this temperature for 5 h. The crystals were filtered off, washed with 50 ml of pre-cooled toluene and dried at 50° C./<30 mbar to afford 34.5 g (69.9%) of cyclopropyl sulfonamide as colorless crystals with a purity of 99.8% (GC, % area).

### EXAMPLE 2

To a solution of 18.3 g (0.25 mol) tert-butylamine and 25 g (0.25 mol) triethylamine in 200 mL of toluene cooled to 10° C. to 20° C. was added 3-chloropropane sulfonyl chloride (36.0 g, 0.21 mol) within 30 to 60 minutes and the resulting mixture was stirred at 15° C. for 30 min. The mixture was treated with 90 mL of 1M hydrochloric acid. The layers were allowed to separate and the aqueous layer was removed. The organic layer was washed with water (1×50 mL). From the organic layer toluene was distilled off and replaced by THF. The resulting mixture (150 mL) was cooled to −25° C. and treated at this temperature with 210 g (0.49 mol) of BuLi (15% in hexane). After 30 min at −25° C. the mixture was warmed to 0° C. and treated with water (100 mL). The layers were allowed to separate. The organic layer was washed with water (4×100 mL). The combined aqueous layers were treated with 37% hydrochloric acid (32 mL) and then extracted with toluene (2×100 mL). The combined organic layers were concentrated to a residual volume of 200 mL and then washed with water (2×20 mL). The organic layer was concentrated to dryness. The residue was treated with formic acid (135 mL) and water (15 mL) at 80° C. for 7 h. During the entire reaction a slight stream of nitrogen was bubbled through the solution. After complete conversion the mixture was concentrated to dryness. Residual amounts of formic acid were removed by co-evaporation with toluene (2×100 mL). The resulting residue was dissolved in ethanol (60 mL) at 64° C. and treated with 1.0 g of charcoal. The charcoal was filtered off and washed with ethanol (20 mL). The mixture was heated to reflux temperature and concentrated to a residual volume of approximately 40 mL. The solution was then treated within 30 min at 70° C. with toluene (125 mL). The mixture was cooled to −10° C. within 4 h and stirred at this temperature for 5 h. The crystals were filtered off, washed with 40 mL of pre-cooled toluene and dried at 50° C./<30 mbar to afford 18.73 g (75%) of cyclopropyl sulfonamide as colorless crystals with a purity of 99.9% (GC, % area).

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A process for the preparation of cyclopropyl sulfonamide (I) comprising the steps of:
(i) contacting chloropropane sulfonyl chloride (II) with N-tert-butyl amine and a base in a first non-polar solvent to afford N-tert.-butyl-(3-chloro) propyl sulfonamide (III); 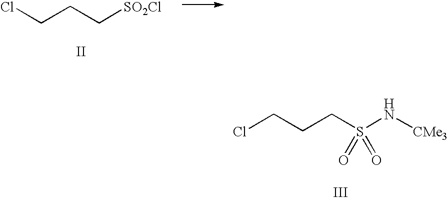
(ii) contacting III with a n-alkyl lithium in a second no-polar solvent to cyclize the 3-chloro-group and afford IV; 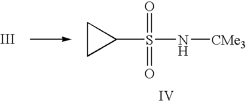
(iii) contacting IV with acid, while passing inert gas through the reaction, to cleave the tert-butyl group and afford I 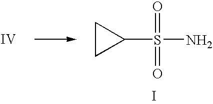
wherein the process is performed without the isolation of III and IV.

2. A process according to claim 1 wherein the organic solvent in step (i) is toluene.

3. A process according to claim 1 wherein the base in step (i) is triethylamine.

4. A process according to claims 1 wherein step (i) is carried out at a reaction temperature from −50° C. to 40° C.

5. A process according to claim 1 wherein N-tert-butyl-(3-chloro) propyl sulfonamide (III) is provided for use in step (ii) as an anhydrous toluene solution after quenching step (i) with aqueous hydrochloric acid, separating the toluene layer from the aqueous layer, washing the toluene layer with water and removing the water by azeotropic distillation.

6. A process according to claim 1 wherein the n-alkyl lithium is n-butyl lithium or n-hexyl lithium.

7. A process according to claim 1 wherein the second organic solvent in step (ii) is a mixture of toluene and tetrahydrofuran.

8. A process according to claims 1 wherein the conversion in step (ii) is carried out at a reaction temperature from −70° C. to 0° C.

9. A process according to claim 1 wherein the cyclopropane sulfonic acid tert.-butylamide (IV) is provided for use in step (iii) as a toluene/tetrahydrofuran solution.

10. A process according to claim 1 wherein the acid used in step (iii) is formic acid or aqueous formic acid.

11. A process according to claim 1 wherein the conversion in step (iii) is carried out at a reaction temperature from 60° C. to 100° C.

12. A process according to claims 1 wherein in step (iii) the inert gas is nitrogen.

13. A process according to claim 1 further comprising the step of crystallizing I from a mixture of toluene and ethanol.
